(19) United States
(12) Patent Application Publication
(10) Pub. No.: US 2022/0350921 A1
(43) Pub. Date: Nov. 3, 2022

(54) SYSTEMS AND METHODS FOR THE SAFE TRANSFER AND VERIFICATION OF SENSITIVE DATA

(71) Applicant: INTELLIJOINT SURGICAL INC., KITCHENER (CA)

(72) Inventors: JOSEPH ARTHUR SCHIPPER, KITCHENER (CA); ANDRE NOVOMIR HLADIO, WATERLOO (CA); JUSTIN AARON MICHAEL, VANCOUVER (CA)

(21) Appl. No.: 17/621,291

(22) PCT Filed: Jun. 18, 2020
(86) PCT No.: PCT/CA2020/050843

§ 371 Date: Dec. 21, 2021

Related U.S. Application Data
(60) Provisional application No. 62/864,677, filed on Jun. 21, 2019.

(51) Int. Cl. G06F 21/62; G06F 21/60; H04L 9/32; G16H 10/60

(52) U.S. Cl. CPC G06F 21/6245; G06F 21/602; H04L 9/3239; G16H 10/60; H04L 2209/88

(57) ABSTRACT

There are provided systems and methods for the safe transfer and verification of sensitive data. Personally identifying information from the sensitive data for an individual is encoded using mapping (e.g. hashing) to have statistical characteristics that limit the encoded information from being considered statistically identifiable. Identical, non-unique encoded output may be generated from multiple individuals from their respective unique personally identifying information. Determining the identity of a person using the encoded output (without any other identifying data) is statistically unlikely. The mapping may be configured such that all possible encoded output (e.g. each respective instance of output in the output space) have a statistically equal chance of being generated.

### FIELD

The following relates to computing devices and computing methods for transforming secure data into a format that is safe for physical or digital transfer, and allowing for the verification to a specific identity.

### BACKGROUND

Various systems and methods are known to generate sensitive health information, known as protected health information (PHI). PHI consists of two aspects: personally identifiable information (PII), and health information (HI). PII is any information that individually, or in combination with other readily accessible information, is statistically likely to be able to be linked to an individual person. HI is any information related to the past, current, or future physical or mental health of a person.

It may be desired to transmit PHI from one location to another. In one example, a surgeon may have some information stored on a server in one physical location, such as a hospital network, but may need access to the information at a home office to perform diagnosis or analysis. In another example, a surgeon may desire to share patient images with a third part for processing, and have the results returned.

This transmission may be digital, or physical. Strict regulations exist around the transmission of PHI to protect the individual it refers to. These regulations are complex and expensive to implement, and may include requirements around the security of data at rest, and in transit (encryption), the physical ability to access the data, and the administrative rules regulating access to the data. In addition to this, there may be regulations surrounding what actions a company or individual must take should and PHI be disclosed to an unauthorized party.

When PHI is transmitted from one location to another, it must be able to be linked to the correct patient. For example, if a pre-operative plan is created for a specific patient for hip surgery, then it must be confirmed during surgery that the plan corresponds to the patient undergoing that hip surgery. Failing to use the correct patient specific plan may result in severe consequences for all parties involved, such as the patient, the surgeon, and the hospital.

The regulations for the safe transfer of data are region specific. In the United States, the regulations are outlined in the Health Insurance Portability and Accountability Act (HIPAA). More specifically, the transfer of digital information is governed by the Privacy Rule (why protecting health information is required), and the Security Rule (how to protect health information). The security rule outlines how PHI must be secured, and includes three classes of protection: technical, physical, and administrative.

### SUMMARY

There are provided systems and methods for the safe transfer and verification of sensitive digital information comprising uniquely identifying information and health information. A first computing device may be configured to encode uniquely identifying information from the sensitive digital information for an individual using a colliding hashing algorithm to produce verifiable identifying information. The verifiable identifying information is encapsulated with the health information to produce verifiable digital information, and provided for use with a second computing device.

A second computing device may be configured with to receive verifiable digital information from a first computing device and test identifying information of a second person, and process the test identifying information using a colliding hashing algorithm to produce test verifiable identifying information.

The uniquely identifying information may comprise one or more of the following: patient name, medical record number, social security number/social insurance number, date of birth, date of surgery, or other uniquely identifying codes. The health information may comprise information related to a surgical procedure. In one example the information may be co-registration information and implant target information for hip surgery. The co-registration information may comprise a set of pre-define points in an anatomic coordinate system. The implant target information may comprise one or more of acetabular implant inclination, acetabular implant anteversion, acetabular implant location, acetabular implant make and model, femoral implant version, femoral implant offset, femoral implant make and model, or bone cut locations.

The colliding hashing algorithm may comprise steps which when executed by a first or second computing device computes verifiable digital information from uniquely identifying information which is statistically likely to be share among multiple individuals in a population (e.g. with different uniquely identifying information), but is statistically unlikely to be identical between two random individuals in a population. The colliding hashing algorithm may comprise a first mapping function which has a statistically insignificant change of output collision (e.g. such as MD5, SHA-1, SHA-2, SHA-256, SHA-3) and a second mapping function to increase the chance of output collision (e.g. such as a binning function or integer modulus function).

The first computing device may be configured to export the verifiable digital information as a QR code image. The first computing device may be further configured to perform preoperative planning and provide the results of planning as health information for use by a second computing device.

The second computing device may be located in an operating room, and may be further configured to receive a preoperative plan form a first computing device and to deliver the preoperative plan using computer aided navigation.

The test identifying information may be derived from another source at the time of the surgical procedure which the other source may be a patient chart, a networked medical database, or a physical or digital image in the operating room.

The second computing device may be configured to provide for display to a user the verifiable identifying information from the verifiable digital information and the test verifiable identifying information to a user for visual comparison, or may be configured to perform the comparison and provide for display to a user the results of the comparison. The second computing device may be further configured to automatically begin a further process based on the results of the comparison such beginning an anatomic registration step.

### BRIEF DESCRIPTION OF THE DRAWINGS

FIG. 1A is a block diagram of a first computing device configured to generate and communicate verifiable identification information from uniquely identifying information and health information in accordance with an example.

FIG. 1B is a block diagram showing a flow of operations in accordance with an example with reference to FIG. 1A.

FIG. 2 is a flow chart of operations in accordance with an example.

FIG. 3A is a block diagram of a second computing device configured to receive and verify verifiable identification information in accordance with an example.

FIG. 3B is a block diagram showing a flow of operations in accordance with an example with reference to FIG. 3A.

The present concept of the invention is best described through certain embodiments thereof, which are described herein with reference to the accompanying drawings, wherein like reference numerals refer to like features throughout. It is to be understood that the term invention, when used herein, is intended to connote the concept underlying the embodiments described below and not merely the embodiments themselves. It is to be understood further that the general concept is not limited to the illustrative embodiments described below and the following descriptions should be read in such light. More than one concept may be shown and described and each may standalone or be combined with one or more others unless stated otherwise.

### DETAILED DESCRIPTION

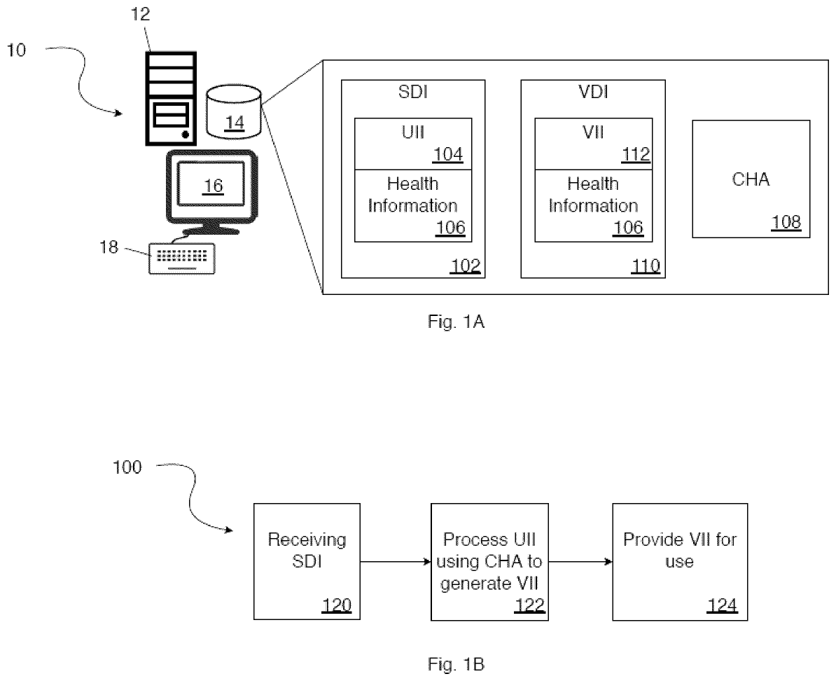

FIG. 1A is a block diagram of a first computing device 10 comprising a processor 12 and memory 14 coupled to a display 16 and input device(s) such as a keyboard 18. Other input devices may include a microphone, pointing device, touchscreen, etc. (not shown). Other output devices not shown may include a speaker, lights, etc. Processor 12 may be configured such as via instructions (and data) stored in memory 14 (or other storage device) which when executed by the first computing device configure the computing device to perform a method 100 shown in a simplified manner in FIG. 1B. The instructions etc. may define a (software) application, or a subset of an application, which may reside on the computing device 10. The computing device 10 may be further configured to perform pre-operative surgical planning such as via software. The instructions may provide a graphical user interface to guide the user to provide input for the operations of method 100. FIG. 1A shows various data and instructions as further described stored in memory 14 and FIG. 1B shows a data flow (e.g. method 100) for processing such data using such instructions.

Method 100 comprises operations such as to perform steps. Operations of method 100 include, at 120, receiving sensitive digital information (SDI) 102. SDI may also be known as PHI as defined according to HIPAA, but may be defined using other terminology in other regions. The source of SDI 102 may be a hospital, a patient, or a third-party such as a planning company. The information may be in a standard format such as DICOM, or may be received in a custom format, such as JSON or CSV, unique to a third-party. The information may be received from a communication to computing device 10, by receiving input thereto via a keyboard, microphone, touch screen, pointing device or other input device, by media such as a CD ROM, flash drive, etc. or retrieval from remote storage such as a server, database, etc.

SDI 102 comprises uniquely identifying information (UII) 104, and health information (HI) 106. Ull 104 may comprise one or more identifying characteristics. Characteristics may be considered identifying individually, such as a social security number (SSN) or a medical records number (MRN). Characteristics may also be considered identifying when combined in plurality, such as name and address, or name and zip code, such that the characteristics may not be individually identifying (multiple individuals may have the same name, or may have the same zip code), but together, are statistically likely to identify a unique individual.

HI 106 may comprise of any data which relates to the health of an individual. HI 106 may comprise a plan for a surgical procedure such as orthopaedic hip surgery or other surgical procedures require health information (not shown). This plan may comprise implant specifications and targets, and information for co-registration between a pre-operative planning coordinate system, and an intra-operative navigation coordinate system.

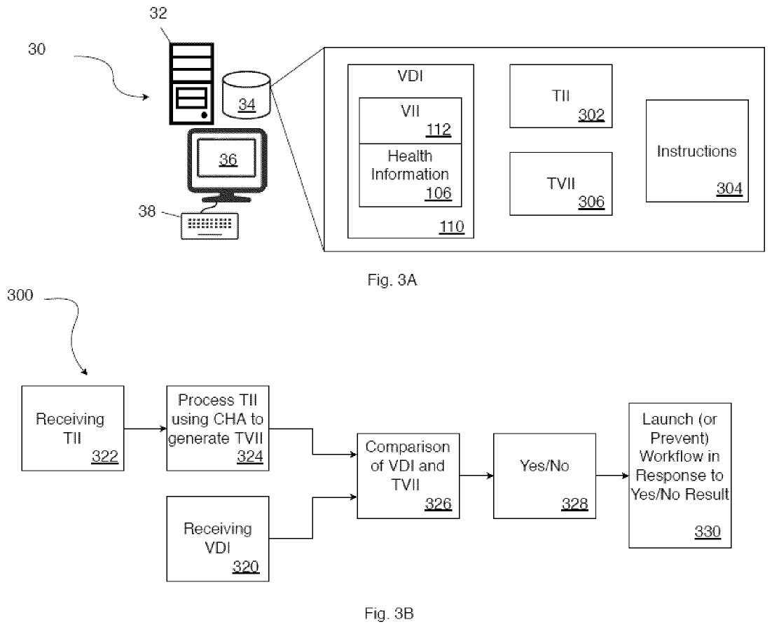

Operations 100 further comprise, at 122, processing the SDI using a colliding hashing algorithm (CHA) 108. CHA 108 may be configured to deterministically transform Ull 104 into verifiable identifying information (VII) 112 comprising text data (which may include alphabetic characters, numeric characters and other symbols, such as punctuation and mathematical symbols and any combination of same). CHA 108 may generate VII 112 to have statistical characteristics that limit the information from being considered statistically identifiable. Identical, non-unique VIls 112 may be generated from multiple individuals from the respective unique Ulls 104 from those individuals. Determining the identity of any person using VII 112 alone (without any other identifying data) would be statistically unlikely. CHA 108 may be configured such that all possible encoded output VII 112 (e.g. each respective instance of output in the output space) have a statistically equal chance of being generated. Operations 100 include, at 124, providing VII 112 for use such as is further described herein below with reference to FIGS. 3A and 3B.

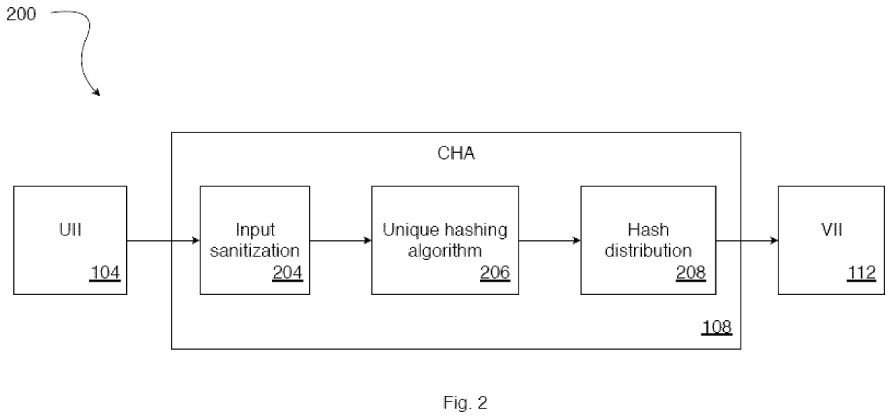

In accordance with an example, CHA 108 comprises multiple steps to perform the transformation such as shown in flow 200 of FIG. 2. A first step 204 comprises input data sanitization whereby multiple input data, encapsulated in Ull 104, are combined and processed to become uniform and expressed in a common format. In an example, the identifying characteristics of name and date of birth John Smith, born Dec. 21, 1965, may be transformed to a string of text “johnsmith19651221”, under the constraints that there are no spaces, all characters are lower case, and the date appears a single number of eight digits. Removing spaces, using all lower case and enforcing a common date format may remove data entry differences that are not intended to reference a different person. In this way, when a mapping function is applied to a specific person's Ull, it will consistently map to the same output. Consistency is important when performing a comparison as described further below.

A second step 206 comprises using a (first) mapping function to calculate a mapping output value from the sanitized input data. Preferably the mapping function is a one way function. A good function also preferably outputs a significantly different value, even for small changes made to the input. Further a good mapping function avoids collision. The function may be a (cryptographic) hash function such as MD5, or alternatively SHA-1or SHA-256, a fingerprinting function (e.g. a high-performance hash function such as Rabin's algorithm), a checksum function, etc. It may be assistive to define an input string of Ull 104 with a minimum length (e.g. a number of characters) such as by padding fields such as first name, surname, etc. In the example, the string “johnsmith19651221” is hashed to the hexadecimal string “3968d22aa819132722ae3526292599df”.

A third step 208 comprises a step wherein the hash is binned (e.g. using a second mapping function) into a finite small number of bins. In some instances, limitations of the computing device (e.g. working with very large numbers) may require that the hash string is first reduced by utilizing only a short substring. In an example, the last 8 characters “292599df” may be used, and first converted to the decimal equivalent number 690330079, and then binned using integer modulus (where the modulo function is the second mapping function, which does not avoid collisions). If there are 128 bins, the number 690330079 is put in the bin with number 95.

Thus the CHA 108 transforms Ull 104 using a first mapping function to encode the Ull into a deterministic value to obscure the Ull information but which encoded Ull may identify the individual by distinguishing them from all other individuals and then uses a second mapping algorithm to transform the encoded Ull to a final code (VII 112) that ensures sufficient collisions so that the final code is evenly distributed among all possible final codes.

It will be apparent that other first mapping functions or second mapping functions may be employed. A (cryptographic) hashing function may be preferred as a good hashing function for the first mapping function because of the properties such hashing functions provide, such as the one way nature of the encoding and the fact that small changes in input become large changes in output. Further after the binning step (e.g. using the modulo function, a type of hash reduction function herein), due to the apparent pseudorandom nature of hashes each of the possible bins (e.g. 0 to 127 per the example) are equally likely to be generated. The modulo function is convenient as it is easy to increase or decrease the number of bins (i.e. change the size of the output space for the encoded output) to adjust for risk as described further below.

Method 100 may then combine VII 112 with HI 106 to produce verifiable digital information (VDI) 110. Method 100 may be enabled to provide VDI 110 for communication with another system (comprising a second computing device). In one example, this communication may be through an internet server. In other implementations, this communication may utilize an email client or a USB storage device or other storage media, etc.

Method 100 may alternatively be configured to provide VDI 110 for communication using a standard physical encoding, such as a quick response (QR) code, which may enable the physical transfer on paper or via a display screen each using an optical transfer (e.g. for reading into a second computing device through a camera).

Regional regulations may restrict the transmission of SDI 102 unless subject to strict rules, because SDI 102 can link HI 106 directly to an individual using Ull 104. These same regulations may instead permit the transmission of VDI 110, because there is no specific information which can link directly to a single individual.

FIG. 3A is a block diagram of a second computing device 30 comprising a processor 32 and memory 34 coupled to a display 36 and input device(s) such as a keyboard 38. Other input devices may include a microphone, pointing device, touchscreen, etc. (not shown). Other output devices not shown may include a speaker, lights, etc. Processor 32 may be configured such as via instructions (and data) stored in memory 34 (or other storage device) which when executed by the second computing device 30 configure the computing device to perform a method 300 shown in a simplified manner in FIG. 3B. These instructions etc. may define a (software) application or a subset of an application, which may reside on the computing device 30. The second computing device 30 may be configured to perform intra-operative surgical navigation. The instructions may provide a graphical user interface to guide the user to provide input for the operations of method 300 and to provide to the user feedback on the results of method 300. FIG. 3A shows various data and instructions as further described stored in memory 34 and FIG. 3B shows a data flow (e.g. method 300) for processing such data using such instructions.

Method 300 comprises operations such as to perform steps. Operations of method 300 include, at 320, receiving VDI 110 from computing device 10, comprising HI 106, which may be a pre-operative plan for an individual, and VII 112 which is derived using CHA 108 from Ull 104 corresponding to that same individual.

When health information is provided to a system to provide treatment to an individual, it is necessary to ensure that the correct health information is used. Providing treatment to an individual based on incorrect health information can have negative consequences, possibly resulting in harm or death to an individual. It may therefore be desirable for VDI 110 to be verified to correspond to the individual being treated intra-operatively.

For verification, method 300 may also, at 322, receive test identifiable information (TII) 302, which is known (e.g. expected) to correspond to the individual undergoing treatment. In one example, TII 302 may be known to correspond to the individual because the information is derived from the individual's “chart” which is read aloud prior to treatment. TII 302 is defined in an identical format to Ull 104.

At step 324, method 300 is configured to process TII 302 using CHA (as part of instructions 304) to create test verifiable identifying information (TVII) 306. The CHA in instructions 304 may be configured to be identical to CHA 108 in method 100. Method 300, at step 326, compares VII 112 to TVII 306 using a comparison algorithm (part of instructions 300) to determine if VDI 110 corresponds to the individual undergoing treatment based and, at step 328, provides a result of the comparison, which may be presented via display 36 and/or other output device (e.g. speaker, bell, light etc. all not shown).

Since there may be multiple identifying characteristics which may generate the same VII 112, it may also be possible that VII 112 corresponding to a different individual may be equal to TVII 306, which may result in the incorrect treatment of the individual. CHA 108 may be configured to generate a VII 112 such that the likelihood of incorrect identifying information also generating an equivalent TVII 306 is lower than an acceptable risk threshold yet still satisfy the desire that the VII 112 is statistically unlikely to identify any individual. As noted above, adjusting the second mapping function such as by adjusting the number of bins via the modulo function may be performed to vary the rate of collisions.

In one example, an acceptable risk threshold may be determined such that that one out of every 10,000 individuals may be provided incorrect treatment. It may also be determined that the likelihood of the incorrect VDI 110 being received by method 300 to be one percent, or one out of 100. The incorrect VDI 110 may be received by method 300 due to human error. If VII 112 was generated with the properties that there were 128 unique possible VIIs, and each was statistically likely to be generated, then there would only be a one out of 128 chance (0.78%) that VII 112 generated from one individual would be equivalent to TVII 306 generated from a different individual. In combination, the likelihood of the incorrect VDI 110 being provided to method 300, and the likelihood that VII 112 would also be equivalent to TVII 306, results in a likelihood that only 1 out of every 12,800 individuals would receive incorrect treatment, which is a lower likelihood than the defined acceptable risk.

Alternatively or in addition, method 300 may be configured to simultaneously display TVII 306 and VII 112 for comparison by a user by visual inspection using display 36, or other means.

Method 300 (e.g. at 330) may be configured to automatically launch (or prevent the launch of) a further process such as a pre-defined workflow based on results at step 328. In one example, method 300 may proceed to a co-registration step if the result at step 328 identifies a positive match between TVII 306 and VII 112.

Practical implementation may include any or all of the features described herein. These and other aspects, features and various combinations may be expressed as methods, apparatus, systems, means for performing functions, program products, and in other ways, combining the features described herein. A number of embodiments have been described. Nevertheless, it will be understood that various modifications can be made without departing from the spirit and scope of the processes and techniques described herein. Accordingly, other embodiments are within the scope of the following claims.

Throughout the description and claims of this specification, the word “comprise” and “contain” and variations of them mean “including but not limited to” and they are not intended to (and do not) exclude other components, integers or steps. Throughout this specification, the singular encompasses the plural unless the context requires otherwise. In particular, where the indefinite article is used, the specification is to be understood as contemplating plurality as well as singularity, unless the context requires otherwise. Herein, “A and/or B” means A or B or both A and B.

Features, integers, characteristics, etc. described in conjunction with a particular aspect, embodiment or example are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings).

1. A method for the secure transfer and verification of sensitive digital information, the method for performing by a first computing device and comprising:
transforming sensitive digital information (SDI) into verifiable digital information (VDI) for the purpose of exporting the VDI to a second computing device, wherein:
the SDI comprises uniquely identifying information (UII) of a first person, and health information of the first person;
the VDI comprises verifiable identifying information (VII) derived from the UII, and the health information from the first person; and
the VII is derived from the Ull using a colliding hashing algorithm (CHA); and
exporting the VDI for receiving by the second computing device, which second device is configured to verify the health information relates to the first person.

2. (canceled)

3. The method of claim 1, wherein the UlI comprises one or more of:
patient name;
medical record number;
social security number/social insurance number;
date of birth;
date of surgery; or
other uniquely identifying codes.

4. The method of claim 1, wherein the health information comprises a patient specific plan for a surgical procedure comprising of co-registration information and implant target information for a hip surgery.

5. The method of claim 4, wherein the co-registration information is a set of pre-defined points in an anatomic coordinate system.

6. The method of claim 4, wherein the implant target information comprises one or more of:
acetabular implant inclination;
acetabular implant anteversion;
acetabular implant location;
acetabular implant make and model;
femoral implant version;
femoral implant offset;
femoral implant make and model; and
bone cut locations.

7. The method claim 1, wherein the CHA computes a VII that is shared among multiple individuals in a population, but is statistically unlikely to be identical between two random individuals.

8. The method claim 1, wherein the CHA comprises a first mapping function that has a statistically insignificant chance of output collision, and a second mapping function to perform a reduction and that increases the chance of collision.

9. The method of claim 8, wherein the first mapping function comprises any one of the hashing functions:
MD5;
SHA-1;
SHA-2;
SHA-256; and
SHA-3.

10. The method of claim 8, wherein the second mapping function operates to map output from the first mapping function into one of a small integer number of bins.

11. The method of claim 1, wherein the VDI is exported as a Quick Response (QR)code image.

12. The method of claim 1, wherein the first computing device is further configured to perform pre-operative planning.

13. The method claim 1, wherein the second computing device is located in an operating room, and the second computing device is further configured to deliver a pre-operative plan using computer aided navigation.

14. A method for the secure transfer and verification of sensitive digital information, the method for performing by a second computing device and comprising:
receiving verifiable digital information (VDI), exported from a first computing device, and test identifying information (TII) of a second person; and
defining test verifiable identifying information (TVII) for the purpose of verifying that the identity of the second person matches the identity of the first person, wherein:
the VDI comprises verifiable identifying information (VII) derived from uniquely identifying information (UII) of a first person using a colliding hashing algorithm (CHA), and further comprises health information from the first person;
the TII comprises uniquely identifying information for the second person and has a same format as the UII; and
the TVII is derived from the TII using the CHA; and
providing the TVII for comparison with the VDI to verify the identity related to the health information

15. The method of claim 14, wherein the second computing device is configured to display the VII and the TVII to a user for visual comparison.

16. The method of claim 14, further configured to compare the VII and TVII and display to a user the results of the comparison.

17. The method of claim 16, wherein the second computing device is further configured to automatically begin a further process based on the result of the comparison.

18. (canceled)

19. (canceled)

20. The method of claim 14, wherein the TII of the second person is derived from another source at the time of an operation on an anatomy of the second person, which other source comprises one of:
a patient chart;
a networked medical database; and
a physical or digital image in the operating room.

21. The method of claim 14, wherein:
the CHA computes TVII that is shared among multiple individuals in a population, but is statistically unlikely to be identical between two random individuals;
the CHA comprises a first mapping function that has a statistically insignificant chance of output collision, and a second mapping function to perform a reduction and that increases the chance of collision.

22. The method of claim 21, wherein:
the first mapping function comprises any one of the hashing functions: MD5; SHA-1; SHA-2; SHA-256; and SHA-3; and
the second mapping function operates to map output from the first mapping function into one of a small integer number of bins.
